(19) United States
(12) Patent Application Publication
(10) Pub. No.: US 2024/0385152 A1
(43) Pub. Date: Nov. 21, 2024

(54) SUGAR CHAIN ANALYSIS METHOD

(71) Applicants: JAPANESE FOUNDATION FOR CANCER RESEARCH, Tokyo (JP); Shimadzu Corporation, Kyoto (JP)

(72) Inventors: Koji UEDA, Tokyo (JP); Yoshimi HAGA, Tokyo (JP); Masaki YAMADA, Kyoto (JP)

(73) Assignees: JAPANESE FOUNDATION FOR CANCER RESEARCH, Tokyo (JP); Shimadzu Corporation, Kyoto (JP)

(21) Appl. No.: 18/287,424

(22) PCT Filed: Jun. 28, 2022
(86) PCT No.: PCT/JP2022/025780

§ 371 Date: Oct. 18, 2023

(30) Foreign Application Priority Data
Aug. 27, 2021 (JP) 2021-138767

(51) Int. Cl. G01N 30/72; C12Q 1/34; G01N 30/02; G01N 30/88; H01J 49/42

(52) U.S. Cl. CPC G01N 30/7233; C12Q 1/34; G01N 30/88; H01J 49/42; G01N 2030/027; G01N 2030/8813

(57) ABSTRACT

A sugar chain analysis method is a method for analyzing a sugar chain structure bound to an antibody, the method including: a releasing step of adding a sugar cleavage enzyme to a sample containing an antibody, so that a sugar chain bound to the antibody is specifically released to obtain a free sugar chain; a recovery step of recovering the free sugar chain by affinity purification; a hydrophobization step of hydrophobizing the free sugar chain to obtain a hydrophobized sugar chain; and a measurement step of performing supercritical liquid chromatography and tandem mass spectrometry in this order on the hydrophobized sugar chain.

### TECHNICAL FIELD

The present invention relates to a sugar chain analysis method.

### BACKGROUND ART

Sugar chains of various structures are non-uniformly bound to an antibody, and the function and effect of the antibody is influenced by the types and proportions of sugar chains. For this reason, it is important to analyze a sugar chain bound to an antibody.

As a relevant analysis method, a method has been heretofore used in which a sugar chain cleaved from an antibody is fluorescently labeled, and analyzed by high performance chromatography (fluorescent HPLC method) (Non-Patent Document 1). In recent years, methods for analyzing sugar chains by matrix-assisted laser desorption-ionization mass spectrometry, gas chromatography mass spectrometry or the like have been reported (Non-Patent Document 2). As an exceedingly sensitive analysis method, a capillary electrophoresis-mass spectrometry has been reported (Non-Patent Document 3).

### PRIOR ART DOCUMENTS

### Non-Patent Documents

Non-Patent Document 1: mAbs 7:1, 167-179; January/February 2015; Published with license by Taylor & Francis Group, LLC

Non-Patent Document 2: Nature Protocols, 2007 Vol. 2, No. 7 1585-1602.

Non-Patent Document 3: Nature Communications 2019 10:2137.

### SUMMARY OF THE INVENTION

### Problems to be Solved by the Invention

However, the analysis methods of Non-Patent Documents 1 and 2 enable a sugar chain concentration to be detected only at a level of subpicomoles (for example, 0.03 picomoles) or greater, and is not sufficient in sensitivity. On the other hand, in the analysis method of Non-Patent Document 3, it is possible to detect a sugar chain at an attomole level, but in capillary electrophoresis, setting of electrophoresis conditions is delicate and difficult. Therefore, other options are required as a sensitive sugar chain analysis method.

An object of the present invention is to provide a method for analyzing a sugar chain of an antibody with ease and high sensitivity.

### Means for Solving the Problems

An analysis method according to a first aspect of the present invention is a method for analyzing a sugar chain structure bound to an antibody, the method including: a releasing step of adding a sugar cleavage enzyme to a sample containing an antibody, so that a sugar chain bound to the antibody is specifically released to obtain a free sugar chain; a recovery step of recovering the free sugar chain by affinity purification; a hydrophobization step of hydrophobizing the free sugar chain to obtain a hydrophobized sugar chain; and a measurement step of performing supercritical liquid chromatography and tandem mass spectrometry in this order on the hydrophobized sugar chain.

### Effect of the Invention

The analysis method according to the first aspect enables a sugar chain of an antibody to be analyzed with ease and high sensitivity.

### BRIEF DESCRIPTION OF THE DRAWINGS

FIG. 1 shows a block diagram of an analysis method according to a first aspect.

FIG. 2 shows an analysis apparatus for use in the analysis method according to the first aspect.

FIG. 3 shows structural formulae and schematic diagrams of a sugar chain to be analyzed and a sugar chain obtained by acetylation thereof.

FIG. 4 shows a MRM chromatogram of an antibody (bevacizumab) measured in Example 1.

FIG. 5 shows a chromatogram of the antibody (bevacizumab) measured in Example 1 (amount of antibody: 10 amol).

FIG. 6 shows a graph showing relationships between the type of sugar chain structure and the relative abundance thereof among lots of the antibody (bevacizumab).

### MODE FOR CARRYING OUT THE INVENTION

### 1. First Aspect

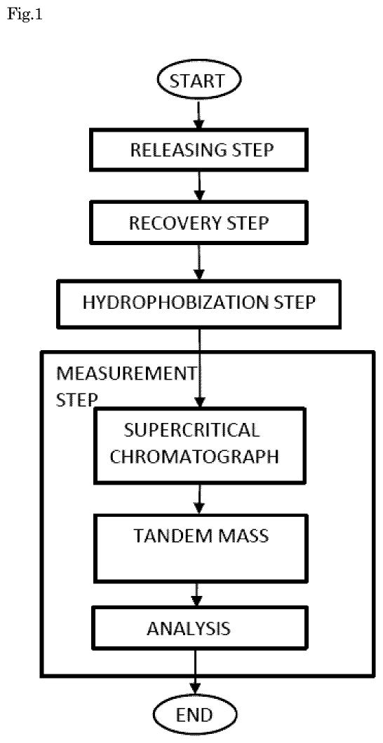

In the analysis method according to the first aspect of the present invention, a releasing step, a recovery step, a hydrophobization step and a measurement step are carried out in this order as shown in FIG. 1. Hereinafter, the steps will be described in detail.

### (Releasing Step)

In the present step, a sugar cleavage enzyme is added to a sample to specifically release a sugar chain bound to the antibody.

First, a sample containing an antibody is prepared. The antibody may be either monoclonal or polyclonal antibody. Examples of the monoclonal antibody include human antibodies such as ramucirumab, nivolumab, panitumumab, ofatumumab, golimumab, ipilimumab and adalimumab; humanized antibodies such as trastuzumab, bevacizumab, tocilizumab, trastuzumab-DM1, omalizumab, mepolizumab, gemtuzumab, palivizumab, ranibizumab, certolizumab, ocrelizumab, mogamulizumab, eculizumab, tricizumab and mepolizumab; chimeric antibodies such as rituximab, cetuximab, infliximab and basiliximab: and murine antibodies.

Antibodies are generally prepared in the form of samples contained in preparations such as intravenous injection preparations and subcutaneous injection preparations; and biological samples such as blood, serum, plasma, saliva, nasal secretions, intestinal secretions, tissues and cells. These preparations and biological samples may be subjected to pretreatment for removal of impurities or the like if necessary.

Sugar chains such as N-bound sugar chains (N-type sugar chains) and O-bound sugar chains (O-type sugar chains) are bound to the antibody. A desired antibody region where a sugar chain is bound is selected, and the sugar chain are released from the antibody region in accordance with a known method.

As a sugar cleavage enzyme, any known enzyme can be used as long as it can cleave a sugar chain from an antibody. Examples thereof include N-glycosidase, O-glycosidase, and endoglycoceramidase. Specifically, when a N-type sugar chain bound to asparagine No. 297 (Asn-297) in a Fc region of an antibody is released, an enzyme such as peptide-N-glycosidase F (PNGase F), peptide-N-glycosidase A (PNGase A) or endo-β-N-acetylglucosaminidase (Endo M) may be used. For the addition amount and the reaction conditions, a protocol accompanying a catalyst to be used may be followed, and for example, denaturation or incubation by heating is appropriately performed in a buffer containing an antibody and a sugar cleavage enzyme.

In this way, a free sugar chain is obtained. Preferably, a N-type sugar chain that has been bound to asparagine No. 297 in the Fc region of the antibody is obtained. Here, the free sugar chain is obtained in a state of a digestion mixture containing an antibody (the sugar chain has been cleaved), a sugar cleavage enzyme, and impurities.

### (Recovery Step)

In the present step, the free sugar chain is recovered by affinity purification. That is, the free sugar chain is isolated or fractionated.

In the affinity purification, the sugar chain is adsorbed to a carrier, the carrier with the sugar chain is washed, and the sugar chain is then eluted in a desired solvent. Since the sugar chain is hydrophilic and the antibody is hydrophobic in general, a carrier whose surface is hydrophilic may be used. Examples of the carrier include hydrophilic beads such as sepharose beads, carboxyl beads, dextran beads and silica beads. Specific examples thereof include Sepharose CL-4B beads (manufactured by SIGMA-Aldrich Co. LLC), and Dynabeads M-270 and M 280 (manufactured by Thermo Fisher Scientific). Examples of the solvent for eluting the sugar chain include aqueous solvents such as water. After purification, the free sugar chain is dried if necessary. The affinity purification in the present invention also includes solid phase extraction using a graphite carbon column or the like.

In this way, the free sugar chain is recovered. Preferably, a N-type sugar chain that were bound to asparagine No. 297 in the Fc region of the antibody is isolated. Here, the isolated free sugar chain may be a free sugar chain alone, or in a state of being contained in an organic solvent.

### (Hydrophobization Step)

In the present step, the free sugar chain is hydrophobized. For example, the hydroxyl group of the free sugar chain is modified (protected) with a hydrophobic compound.

Examples of the hydrophobic method include acylation such as acetylation, propionylation, butyrylation and benzoylation; alkoxymethylation such as methoxymethylation; silylation such as trimethylsilylation, triethylsilylation and t-butyldimethylsilylation; and tritylation. From the viewpoint of adequate retention on a stationary phase in supercritical fluid chromatography, acylation is preferable, and acetylation is more preferable.

The hydrophobic compound reacts with the hydroxyl group of the sugar chain to turn the hydroxyl group into a hydrophobic group, and can be selected from known acylating agents, alkoxymethylation agents, silylation agents, tritylation agents, and the like.

In the case where acetylation is performed, examples of the acetylation agent which is a hydrophobic compound include acetic anhydride, acetyl chloride, acetylimidazole, N-acetylsuccinimide, acetylimidoacetate, and acetylimidazole. In acetylation, a catalyst such as pyridine, triethylamine, sodium acetate, sodium carbonate, sodium bicarbonate, potassium carbonate, imidazole, sodium hydride, sodium methoxide or sulfuric acid may be added if necessary. The acetylation reaction may be carried out under conditions of, for example, 20 to 80° C. and 1 to 10 hours.

In this way, a hydrophobized sugar chain is obtained. Preferably, an acetylated sugar chain is obtained. By hydrophobization, the sugar chain can be dissolved in a supercritical fluid and easily retained in a stationary phase in SFC described later. That is, hydrophobization enables measurement in SFC.

### (Measurement Step)

In the present step, supercritical fluid chromatography (SFC) and tandem mass spectrometry (MS/MS) are performed in this order using the hydrophobized sugar chain as a measurement sample.

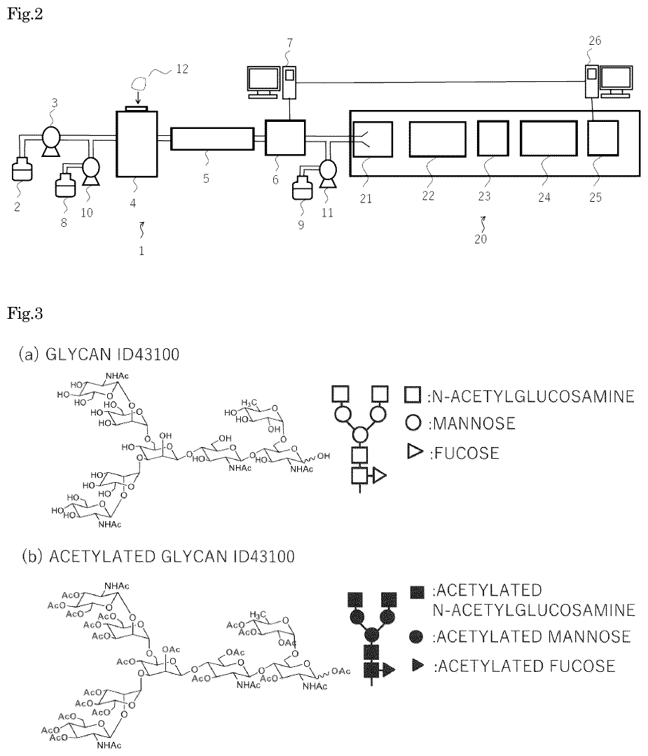

First, SFC is performed on the hydrophobized sugar chain. SFC is chromatography in which a measurement sample is separated using a supercritical fluid as a mobile phase. As shown in FIG. 2, an apparatus 1 for use in SFC includes at least a mobile phase container 2, a pump 3, a sample introduction unit 4, a column 5, a detection 20 unit 6 and a computer 7. It includes a modifier container 8, a makeup solvent container 9, pumps 10 and 11 for delivering their contents and the like if necessary.

Examples of the fluid used for the mobile phase include CO₂, NH₂, N₂O, H₂O, C₃H₈, C₆H₁₄, CH₃OH, C₂H₅OH, and C₆H₅CH₃. CO₂ is preferable from the viewpoint of ease of transition to a supercritical state, suppression of impacts on the human body, and the like.

The stationary phase is not limited as long as it can retain and separate the hydrophobized sugar chain, and examples thereof include carriers having a hydrophobic group. Examples of the hydrophobic group include a phenyl group (−C₆H₅), an octadecyl group, a carbamoyl group, an aminopropyl group, a cholesteryl group, a pentabromobenzyl group, a pyrenylethyl group, a pyridinyl group, a triazole group, and a 3-chloro-4- methylphenyl group. Among them, a substituted or unsubstituted phenyl group is preferable, and an unsubstituted phenyl group (−C₆H₅) is particularly preferable. This improves interaction with a hydrophobized sugar chain, particularly an acetylated sugar chain, so that it is possible to retain and separate the hydrophobized sugar chain. Since the separation completion time can be set to a short time of about 3 to 7 minutes, the time can be significantly reduced in performing high-throughput screening.

Examples of the carrier in which the hydrophobic group is modified include silica beads, acrylic beads, and agarose beads.

Examples of the column having such a stationary phase include Shim-pack US series (registered trademark) manufactured by Shimadzu Corporation, CHIRAL PAK series (registered trademark) manufactured by Daicel Corporation, and Cellulose-C series manufactured by YMC. CO., LTD.

The modifier is not limited as long as solubility in the fluid in the hydrophobized sugar chain can be adjusted, and examples thereof include organic solvents such as methanol, ethanol, 2-propanol, hexane, acetone, isooctane, acetonitrile, tetrahydrofuran, ethyl acetate, dichloromethane, chloroform, 1,4-dioxane, diethyl ether, diisopyl ether, dimethylformamide, and dimethyl sulfoxide.

For example, an acid such as formic acid or acetic acid; a salt such as ammonium formate or ammonium acetate; or a base such as diethylamine or ammonia may be added to the modifier. A carboxylic acid or a salt thereof is preferable from the viewpoint that the hydrophobized sugar chain is well dissolved in the fluid.

In SFC measurement, first, a free sugar chain (measurement sample) 12 is put in the sample introduction unit (automatic sampler) 4. The free sugar chain 12 is mixed with and dissolved in a supercritical fluid and a modifier delivered from the mobile phase container 2 and the modifier container 8 by pumps 2 and 10, and then delivered to the column 5. In the column 5, the free sugar chain is discharged while being separated to a degree according to an elution time by interaction with the stationary phase. The separated free sugar chain is detected by the detection unit 6 and processed into a data form such as a chromatogram by the computer 7. Thereafter, the free sugar chain is subjected to tandem mass spectrometry.

Preferably, a makeup solvent is added to the free sugar chain (specifically, a fluid containing the free sugar chain) having passed through the stationary phase of the column 5. That is, the makeup solvent is delivered from the makeup solvent container 9 by the pump 11, and the free sugar chain and the makeup solvent are mixed on the downstream side of the column 5. This enables improvement of sensitivity in next tandem mass spectrometry. Examples of the makeup solvent include an organic solvent containing a carboxylic acid or a salt thereof. The salt containing the carboxylic acid salt is preferable. Examples of the carboxylic acid include formic acid and acetic acid, and examples of the carboxylic acid salt include ammonium formate and ammonium acetate. Examples of the organic solvent include organic solvents exemplified for the modifier.

Subsequently, tandem mass spectrometry is performed on the free sugar chain subjected to SFC. Tandem mass spectrometry is an analysis method in which at least two mass spectrometries are performed in series, the method including segmenting ions selected by first mass spectrometry, and detecting the fragmented ions by second mass spectrometry. An apparatus for use in tandem mass spectrometry includes at least two mass spectrometer, and a fragment generation unit disposed therebetween. More specifically, as shown in FIG. 2, a tandem mass spectroscope 20 is located on the downstream side of the SFC apparatus 1, and includes an ionization unit 21, a first mass spectrometer 22, a fragment generation unit 23, a second mass spectrometer 24, a detection unit 25, and a computer 26. The ionization unit 21 is configured such that the free sugar chain discharged from the SFC apparatus 1 can be directly introduced into the ionization unit 21. The computer 26 is connected to the computer 7 of the SFC apparatus 1, so that chromatogram results obtained by the SFC apparatus 1 can be shared, compared with results from the detection unit 25, and analyzed.

Examples of the first or second mass spectrometry for use in tandem mass spectrometry include quadrupole mass spectrometers, magnetic field sector mass spectrometers, time-of-flight mass spectrometers, ion trap mass spectrometers, and ion cyclotron resonance mass spectrometers. Hybrid mass spectrometry using two different mass spectrometers is also included in the tandem mass spectrometry in the present invention.

Examples of the process carried out by the ionization unit include electron ionization (EI), electrospray ionization (ESI), and atmospheric pressure chemical ionization (APCI).

Examples of the process carried out by the fragment generation unit include collision induced dissociation (CID), electron transfer dissociation, electron capture dissociation, infrared multiphoton dissociation, ultraviolet photon dissociation, and surface induced dissociation.

Specific examples of the tandem mass spectroscope include triple quadrupole mass spectrometers, quadrupole time-of-flight mass spectrometers (Q-TOF MS), quadrupole ion trap mass spectrometers (Q-IT MS), and tandem time-of-flight mass spectrometers. A triple quadrupole mass spectrometer is preferable from the viewpoint of high selectivity of sugar chains to be analyzed, simple operation, and enabling more sensitive measurement.

In tandem mass spectrometry, the free sugar chain delivered from the SFC apparatus 1 is ionized by the ionization unit 21. Among the ionized free sugar chains, only ionized free sugar chains having a specific m/z value (precursor ions) are introduced into the fragment unit 23 by the first mass spectrometer 22. In the fragment portion 23, the specific ionized free sugar chain is decomposed into a secondary ion (product ion) by a dissociation process such as collision with an inert gas. Among the product ions, only product ions having a desired m/z value (a value smaller than m/z of the precursor ion) are then introduced into the detection unit 24 by the second mass spectrometer 23, detected, and analyzed by the computer 25.

Here, in the first mass spectrometer (Q1 transition), information of all types of precursor ions assumed to be detected (e.g., structure of hydrophobized sugar chain, and m/z) is input in advance. Specific examples thereof are given in Table 1 described in examples below. The m/z value of the precursor ion to be introduced into the fragment generation unit may be set within the range of, for example, 500 or more, preferably 700 or more, and for example, 2,000 or less, preferably 1,500 or less. When a sugar chain enzymatically cleaved with Asn-297 is used, the sugar chain is abundant in sugar chains shown in FIG. 3 (a). For this reason, it is preferable to select and detect at least the m/z value of a precursor ion formed by acetylation of the glycan (specifically, m/z is 1173). That is, it is preferable to select a precursor ion having m/z value of 1173 in the first mass spectrometer. This enables detection of the sugar chain of FIG. 3 (a) as a main component.

In the second mass spectrometer (Q3 transition), the m/z value of the precursor ion to be introduced into the detection unit may be set within the range of, for example, less than 500, preferably 250 or less, and for example, 100 or more, preferably 200 or more, from the viewpoint of detecting a fragment of the sugar chain structure. In particular, it is preferable to select at least the m/z value of a product ion formed by fragmentation of the acetylated free sugar chain (e.g., [HexNc+Ac—2H₂O]⁺: an oxonium ion of acetylated and dehydrated N-acetylhexosamine) (specifically, m/z=210). That is, it is preferable to detect a product ion having a m/z value of 210.

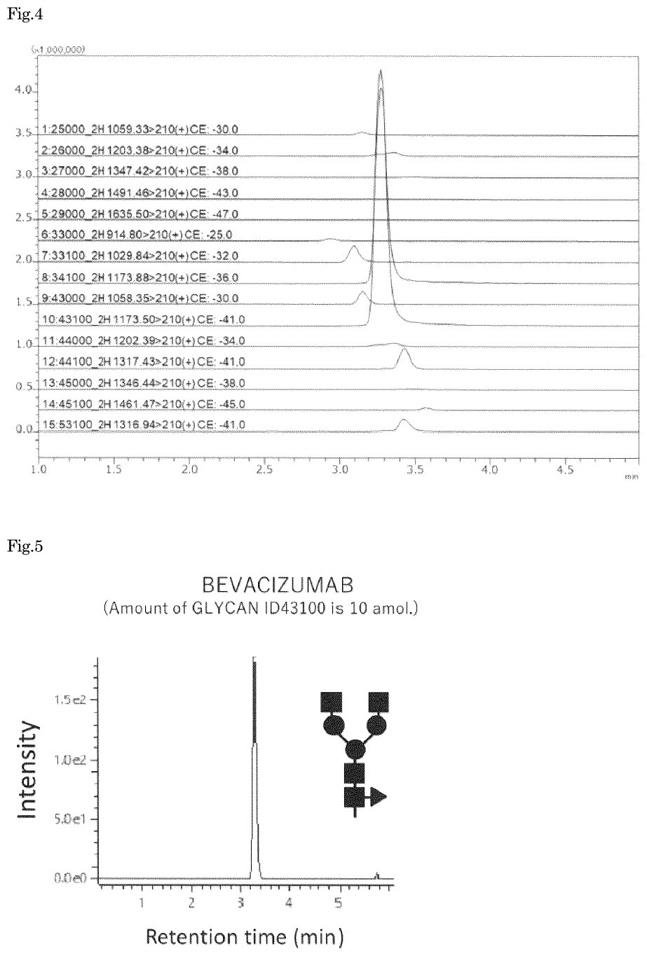

This enables detection of a desired sugar chain structure. Specifically, as shown in FIG. 4 (described in Example 1 below), a multiple reaction monitoring (MRM) chromatogram divided for each desired sugar chain structure can be obtained as mass spectrometric data. That is, a chromatogram of a sugar chain structure corresponding to m/z set by the first and second mass spectrometers can be obtained.

Here, the structure of the sugar chain bound to the antibody is analyzed on the basis of mass spectrometry data obtained by SFC and MS/MS (analysis step). Specifically, the type of the sugar chain bound to the antibody, and the structure and content thereof are determined from the detected m/z and the input information in the obtained MRM chromatogram. These processes can be carried out using known or commercially available software.

From the chromatogram, a desired sugar chain can be detected. Alternatively, for each sugar chain, a calibration curve showing a relationship between the peak intensity of a chromatogram obtained by measurement at a known content (or concentration) and the content, or the like, may be prepared, and consulted to determine the content of a desired sugar chain. Alternatively, a standard substance whose content (or concentration) is known may be added to the measurement sample, followed by calculation of the content of a desired sugar chain from the relative ratio between the peak intensity of the standard substance and the peak intensity of the desired sugar chain.

If necessary, correction may be performed in the calculation of the content. The correction may be performed by a known method, or may be appropriately set. For example, when a difference between the m/z values of precursor ions is less than 1 (for example, the m/z value of one of the sugar chain ions is 1173.3 and the m/z value of the other is 1173.8), a phenomenon may occur in which both the ions are detected at the same time in the first mass spectrometer. In this case, the intensity ratio between the ions is calculated on the basis of data obtained by measurement using a conventional method such as a fluorescence HPLC method, and the peak intensities of the ions are adjusted using the process of the calculation, thereby correcting the contents.

### 2. ASPECTS

Those skilled in the art understand that the exemplary embodiments described above are specific examples of the following aspects.

(Item 1) A sugar chain analysis method according to a first aspect may be a method for analyzing a sugar chain structure bound to an antibody, the method including: a releasing step of adding a sugar cleavage enzyme to a sample containing an antibody, so that a sugar chain bound to the antibody is specifically released to obtain a free sugar chain; a recovery step of recovering the free sugar chain by affinity purification; a hydrophobization step of hydrophobizing the free sugar chain to obtain a hydrophobized sugar chain; and a measurement step of performing supercritical liquid chromatography and tandem mass spectrometry in this order on the hydrophobized sugar chain. This enables provision of a method for analyzing a sugar chain of an antibody with ease and high sensitivity. In particular, a sugar chain bound to an antibody sample at an attomole level can be detected. Accordingly, the non-uniformity of the sugar chain structure can be more reliably analyzed. Since SFC is employed as chromatography, the elution time in chromatography can be set to less than 10 minutes. Accordingly, the measurement time can be significantly reduced as compared to a capillary electrophoresis method (that requires a measurement time of 30 minutes or more). Since the sugar chain discharged from chromatography can be directly delivered to the ionization unit for tandem mass spectrometry, simple, continuous and smooth measurement can be performed. Accordingly, the speed of sugar chain analysis can be increased, so that a high-throughput glycan analysis method can be provided.

(Item 2) In the analysis method according to item 1, the measurement step may include an analysis step of analyzing a structure of a sugar chain bound to the antibody based on mass spectrometry data obtained by supercritical liquid chromatography and tandem mass spectrometry. This enables identification of the type and structure of the sugar chain.

(Item 3) In the analysis method according to item 1 or 2, a stationary phase for use in the supercritical fluid chromatography may have a phenyl group. This enables the free sugar chain to be separated with the free sugar chain reliably retained in the stationary phase in supercritical fluid chromatography.

(Item 4) In the analysis method according to any one of items 1 to 3, the hydroxyl group of the free sugar chain may be modified with a hydrophobic compound in the hydrophobization step. This enables the free sugar chain to be easily dissolved in a fluid for supercritical fluid chromatography, and reliably retained in the stationary phase.

(Item 5) In the analysis method according to any one of items 1 to 4, the hydrophobization may be acetylation. This enables facilitation of hydrophobization (acetylation) of the free sugar chain. In addition, interaction with a stationary phase, in particular, a stationary phase having a phenyl group, can be improved to reliably retain the sugar chain in the stationary phase.

(Item 6) In the analysis method according to any one of items 1 to 5, the measurement step may include adding an organic solvent containing a carboxylic acid or a salt thereof to the free sugar chain having passed through the stationary phase in the supercritical liquid chromatography, and then performing the tandem mass spectrometry. This enables the sugar chain analysis sensitivity to be further more improved.

(Item 7) In the analysis method according to any one of items 1 to 6, a triple quadrupole mass spectrometer may be used in the tandem mass spectrometry. This ensures that a sugar chain to be analyzed can be reliably selected and analyzed with high sensitivity.

### EXAMPLES

The present invention will now be described in detail by giving examples, which should not be construed as limiting the scope of the present invention.

### Example 1

### (Releasing Step)

10 μg of bevacizumab (Avastin: registered trademark, anti-VEGF humanized monoclonal antibody, manufactured by Chugai Pharmaceutical Co., Ltd.) as an antibody was mixed with a Rapid PNGase F reagent (manufactured by New England Biolabs Inc.) in accordance with the manufacturer's protocol. In this way, a digestion mixture was obtained in which N-type sugar chains were released from Asn-297 of the antibody.

### (Recovery Step)

The digestion mixture was prepared in the form of 85% (v/v) acetonitrile, and then dispensed to a filter plate together with 50 μL of Sepharose CL-4B beads (50% slurry, manufactured by SIGMA-Aldrich Co. LLC). Subsequently, it was controlled by 200 μL of water and equilibrated at 85% (v/v) acetonitrile. In this way, the free sugar chains were adsorbed to the beads. The beads with sugar chains were washed twice with a solution containing 85% (v/v) acetonitrile and 0.1% (v/v) trifluoroacetic acid, and then washed twice with 85% (v/v) acetonitrile. Thereafter, the free sugar chains were eluted in 100 μL of water, and vacuum drying was performed.

### (Hydrophobization Step)

The free sugar chains were mixed and reacted with 5 μL of acetic anhydride and 5 μL of dehydrated pyridine under conditions of 50° C. and 4 hours to be acetylated, followed by drying with a centrifugal concentrator. In this way, acetylated sugar chains were obtained.

### (Measurement Step)

The acetylated sugar chains were fractionated such that the amounts were 5,000 fmol, 500 fmol, 50 fmol, 5 fmol, 500 amol, 50 amol, 10 amol and 5 amol in terms of the antibody. That is, the total amount of acetylated sugar chains derived from 10 μg of the antibody were dissolved in methanol, and the solution was appropriately diluted and separated to fractionate the sugar chains such that the ratios to the total amount were ½×10⁶, ½×10⁷, ½×10⁸, ½×10⁹, ½×10¹⁰, ½×10¹¹, 1/1×10¹² and ½×10¹². The thus-obtained solutions were used as measurement samples.

Each of the measurement samples was continuously measured successively with a supercritical fluid chromatography system (NexeraUC manufactured by Shimadzu Corporation) and a triple quadrupole mass spectrometer (LCMS-8050 manufactured by Shimadzu Corporation) in combination (see FIG. 2). The conditions were as follows.

### (1) Supercritical Fluid Chromatography

Mobile phase: CO₂ (99.99% grade, manufactured by Iwatani Corporation)
Modifier: methanol (LC/MS grade)
Injection amount: 2.5 μL
Flow rate: 1.0 mL/min
Column temperature: 40° C.
Column: Shim-pack UC-phenyl (containing C₆H₅ groups, 2.1×150 mm, particle diameter 3 μm: manufactured by Shimadzu Corporation)
Gradient conditions: 10% methanol (0 min)-40% methanol (4 min)-40% methanol (7 min)-10% methanol (7.1 min)-stop (8.5 min).
Makeup solvent: Methanol with 0.1% (v/v) ammonium formate
Flow rate of supplied makeup solvent: 0.1 mL/min

### (2) Triple Quadrupole Mass Spectrometry

Interface voltage: 4000 V
Interface temperature: 225° C.
Heat block temperature: 400° C.
Desolvation temperature: 225° C.
CID gas pressure: 270 kPa (argon gas)

In the Q1 transition, the m/z values of precursor ions were input and set with the sugar chain structures shown in Tables 1 and 2 below so as to cover all possible glycan compositions. In the Q3 transition, the m/z value of the product ion was set to 210, and the dehydrated fragment ion (i.e., [HexNAc+Ac—2H₂O]⁺) of the acetylated sugar chain (GlcNAc) was used as a reporter ion. The collision energy (CE) for detecting m/z=210 was scanned and set so as to maximize the intensity of the fragment ion of the acetylated sugar chain. The acquired data was processed with LabSolutionsLCMS software (manufactured by Shimadzu Corporation) (analysis step).

TABLE 1

| | | precursor | product | |
|---|---|---|---|---|
| | | ion Q1 | ion Q3 | CE |
| Glycan ID | Adduct | (m/z) | (m/z) | (eV) |
| 25000 | M + 2H | 1059.33 | 210 | −30 |
| 26000 | M + 2H | 1203.38 | 210 | −34 |
| 27000 | M + 2H | 1347.42 | 210 | −38 |
| 28000 | M + 2H | 1491.46 | 210 | −43 |
| 29000 | M + 2H | 1635.5 | 210 | −47 |
| 33000 | M + 2H | 914.8 | 210 | −25 |
| 33100 | M + 2H | 1029.84 | 210 | −32 |
| 34100 | M + 2H | 1173.88 | 210 | −36 |
| 43000 | M + 2H | 1058.35 | 210 | −30 |
| 43100 | M + 2H | 1173.5 | 210 | −41 |
| 44000 | M + 2H | 1202.39 | 210 | −34 |
| 44100 | M + 2H | 1317.43 | 210 | −41 |
| 45000 | M + 2H | 1346.44 | 210 | −38 |
| 45100 | M + 2H | 1461.47 | 210 | −45 |
| 53100 | M + 2H | 1316.94 | 210 | −41 |

TABLE 2

| Glycan | |
|---|---|
| ID | Structure |
| 25000 | 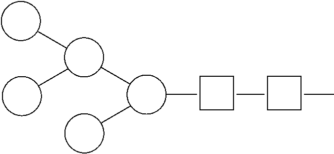 |
| 26000 | 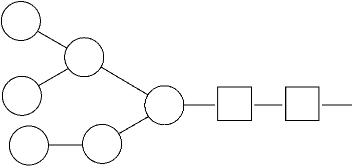 |
| 27000 | 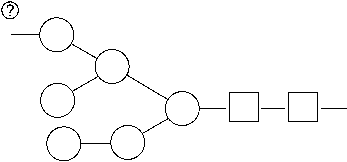 |
| 28000 | 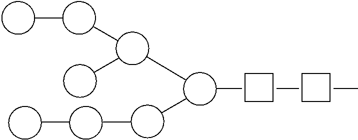 |
| 29000 | 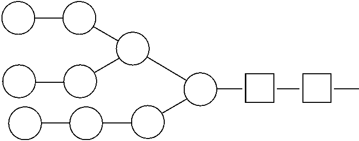 |
| 33000 | 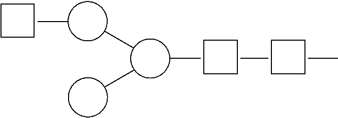 |
| 33100 | 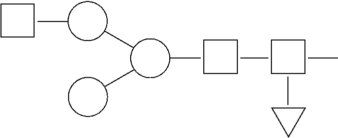 |
| 34100 | 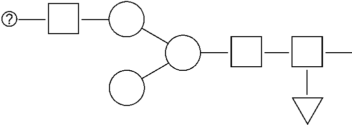 |
| 43000 | 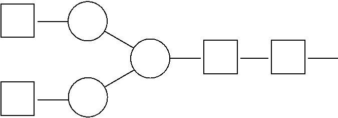 |
| 43100 | 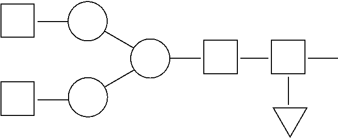 |
| 44000 | 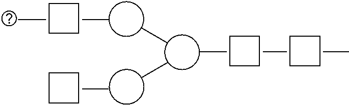 |
| 44100 | 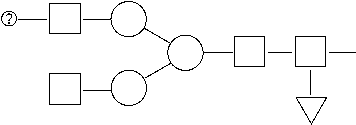 |
| 45000 | 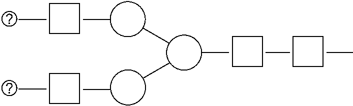 |
| 45100 | 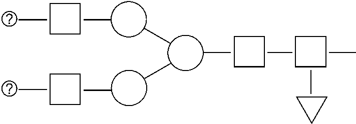 |
| 53100 | 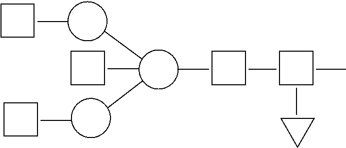 |

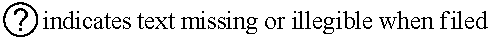

[symbol]

: N-Acetylglucosamine [symbol]
: Fucose [symbol]
: Mannose [symbol]
Galactose

FIG. 4 shows the measurement results when the amount is 500 fmol in terms of the antibody. FIG. 5 shows the measurement results when the amount is 10 amol in terms of the antibody (only the peak for glycan ID43100 is extracted). Table 3 shows the results about a relationship between the amount of the antibody and the peak intensity area at a peak with the highest intensity in the chromatogram.

TABLE 3

| | | |
|---|---|---|
| Amount of | | Variation |
| antibody | Peak area | coefficient(cv)% |

| | | | |
|---|---|---|---|
| 5 | amol | 174.2 | 24.0 |
| 10 | amol | 276.2 | 10.9 |
| 50 | amol | 1225.4 | 9.0 |
| 500 | amol | 10933 | 8.4 |
| 5 | fmol | 122223 | 5.0 |
| 50 | fmol | 1037569 | 3.4 |
| 500 | fmol | 6205217 | 3.9 |
| 5000 | fmol | 25954461 | 2.3 |

From these results, it was found that even when the amount of the antibody was 10 an attomole level amount of 5 amol (lower limit of detection), a sugar chain bound to the antibody was reliably detected by the measurement method of the present example. Thus, this measurement method was considerably superior in sensitivity to common fluorescent HPLC methods in which the detection limit is 0.03 pmol.

### Examples 2 to 5: Analysis of Various Antibodies

Measurement was performed on sugar chains of antibodies in the same manner as in Example 1 except that as the antibody, the following antibodies were used instead of bevacizumab. As a result, even when the amount of the antibody was 5 amol, a sugar chain bound to the antibody was detected as in the case of bevacizumab.

Example 2: Ramucirumab (Cyramza: registered trademark, manufactured by Eli Lilly Japan K.K.)

Example 3: Trastuzumab (Herceptin: registered trademark, manufactured by Chugai Pharmaceutical Co., Ltd.)

Example 4: Nivolumab (Opdivo: registered trademark, manufactured by Ono Pharmaceutical Co., Ltd.)

Example 5: Rituximab (Rituxan: registered trademark, manufactured by Zenyaku Kogyo Company, Limited)

### Example 6: Confirmation of Non-Uniformity of Sugar Chain Structure Between Lots of Antibody

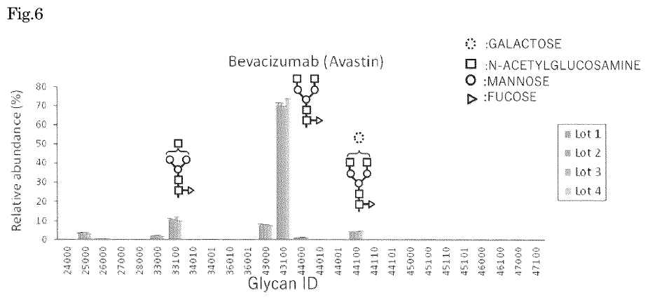

The same procedure as in Example 1 was carried out except that discontinuous four lots (Lots. 1 to 4) of bevacizumab (Avastin: registered trademark, anti-VEGF humanized monoclonal antibody, manufactured by Chugai Pharmaceutical Co., Ltd.) were used as the antibody. FIG. 6 shows a graph showing a relationship between the sugar chain structure (glycan ID) detected at this time and its relative amount. From the graph, non-uniformity between the lots was confirmed because there was a difference between the lots in relative amount of the sugar chain structure (in particular, glycan IDs 33100, 43100 and 44100 whose structures are shown in the graph) bound to the antibody.

### Examples 7 and 8: Comparison of Columns

Measurement in the present invention (Avastin, amount: 500 fmol in terms of antibody) was performed in the same manner as in Example 1 except that the column was changed to those described below. The elution completion time and peak separation in SFC here were observed.

Example 7: Trade name “CHIRALPAK IF-3”, containing a 3-chloro-4- methylphenyl carbamate group, particle diameter: 3 μm, manufactured by Daicel Corporation)

Example 8: Trade name: Cellulose-C, cellulose tris 3,5-dimethylphenyl carbamate, particle diameter: 3 μm, manufactured by YMC Co., Ltd.

Comparison of these results showed that the elution time in each of Examples 7 and 8 was shorter than that in Example 1 with the elution completion time being 2.32 minutes in Example 7 and the elution time being 2.75 minutes in Example 8. However, Examples 7 and 8 were a little poorer in peak separation of the sugar chain than Example 1. Therefore, the column in Example 1 was found to be excellent in both elution rate and peak separation.

### Examples 9 to 10: Comparison of Makeup Solvents

The analysis method of the present invention was carried out in approximately the same manner as in Example 1 except that quadrupole time-of-flight (QTOF) mass spectrometry (LCMS-9030, manufactured by Shimadzu Corporation) was combined instead of the triple quadrupole mass spectrometer, 2 pmol of ramucirumab was used as the antibody, and the makeup solvent was changed to that described below.

The analysis was performed with the makeup solvent set to methanol containing 20 mM ammonium formate (Example 9) or methanol containing 0.1% (v/v) formic acid (Example 9). As a result, at a peak corresponding to a retention time of 3.53 minutes, the peak intensity was 2.25 million counts (height) in Example 9, and the peak intensity was 1.13 million counts (height) in Example 10. This showed that Example 9 was superior in sensitivity.

### DESCRIPTION OF REFERENCE SIGNS

1 supercritical fluid chromatography apparatus
2 mobile phase container
3 pump
4 sample introduction unit
5 column
6 detection unit
7 computer
8 modifier container
9 makeup solvent container
10 pump
11 pump
12 sample
20 tandem mass spectroscope
21 ionization unit
22 first mass spectrometer
23 fragment generation unit
24 second mass spectrometer
25 detection unit
26 computer

1. An analysis method for analyzing a sugar chain structure bound to an antibody, the method comprising:
a releasing step of adding a sugar cleavage enzyme to a sample containing an antibody, so that a sugar chain bound to the antibody is specifically released to obtain a free sugar chain;
a recovery step of recovering the free sugar chain by affinity purification;
a hydrophobization step of hydrophobizing the free sugar chain to obtain a hydrophobized sugar chain; and
a measurement step of performing supercritical liquid chromatography and tandem mass spectrometry in this order on the hydrophobized sugar chain.

2. The analysis step according to claim 1, wherein the measurement step includes an analysis step of analyzing a structure of a sugar chain bound to the antibody based on mass spectrometry data obtained by supercritical liquid chromatography and tandem mass spectrometry.

3. The analysis method according to claim 1, wherein a stationary phase for use in the supercritical fluid chromatography has a phenyl group.

4. The analysis method according to claim 1, wherein a hydroxyl group of the free sugar chain is modified with a hydrophobic compound in the hydrophobization step.

5. The analysis method according to claim 1, wherein the hydrophobization is acetylation.

6. The analysis method according to claim 1, wherein the measurement step includes adding an organic solvent containing a carboxylic acid or a salt thereof to the free sugar chain having passed through the stationary phase in the supercritical liquid chromatography, and then performing the tandem mass spectrometry.

7. The analysis method according to claim 1, wherein a triple quadrupole mass spectrometer is used in the tandem mass spectrometry.
